(19) United States
(12) Patent Application Publication
(10) Pub. No.: US 2021/0402030 A1
(43) Pub. Date: Dec. 30, 2021

(54) SANITIZING DEVICE FOR VENDING MACHINES

(71) Applicant: Richard Eusebio Mattos Vega, Buenos Aires (AR)

(72) Inventor: Richard Eusebio Mattos Vega, Buenos Aires (AR)

(21) Appl. No.: 17/356,878

(22) Filed: Jun. 24, 2021

(30) Foreign Application Priority Data
Jun. 27, 2020 (AR) P20200101830

(51) Int. Cl. A61L 2/24; A61L 2/10; A61L 2/22; G07F 9/10

(52) U.S. Cl. CPC A61L 2/24; A61L 2/10; A61L 2/22; A61L 2202/15; A61L 2202/11; A61L 2202/14; G07F 9/10

(57) ABSTRACT

A sanitizing device having a microcontroller 2 receiving information from the opening sensor 1 of the access status door to the interior of the vending machine activating at least one radiation module 4 or at least one sprayer 5 for a first stage of sanitization of the stored products. The microcontroller 2 operates the control mechanism of the upper doors 7 and the opening and closing mechanism 10 of the front door 9 of a disinfection compartment 6, closing both to start a second disinfection stage that is applied only on the product selected by the consumer when detected by the product sensor 11. The microprocessor 2 opens the front door 9 at the end of the stage, and upon receiving the signal from the empty disinfection compartment 6, triggers the opening and closing mechanism 10, closing the front door 9 and the control mechanism 8 by opening the upper doors 7. The presence detection sent by proximity sensors 3 to the machine to the microcontroller 2, interrupts the cycle of ultraviolet radiation. The microcontroller 2 is linked to a communication interface 14 and a network communication interface 15.

The present invention includes a sanitizing device for vending machines according to the guidelines set out below, which is illustrated with the accompanying figures as a better way to understand the development that is revealed without said description and the afore mentioned figures being limiting of the invention but an aid for its study, understanding and application.

### OBJECT

The current world reality imposes the need to apply methods to take care of the health of the inhabitants; in some areas the movement of people has been restricted and in others the performance of some activities has been limited; and, in general, the distance between people and the use of appropriate means to cover the nose and mouth and to avoid any contagion have been regulated.

It has been recommended that once the purchases are completed, the containers be washed with soap and water or with a mixture of water and alcohol or with a mixture of water and sodium hypochlorite.

This is appropriate when it comes to the products that we purchase to consume at home, however there are some that are purchased to consume on-site, just like those that we obtain from vending machines.

The products obtained from vending machines are very varied since they include sweets, drinks, snacks, cigarettes, sandwiches, etc. and they are elaborated in respective plants.

They then undergo successive handling: they are loaded and transported to a distributor who again loads them, transports them and finally places them on the machines. This determines that we acquire products that have passed through successive hands, that have been stored in different places and that have been carried by different means of transport before being placed in the vending machines.

In this understanding, the inventor has considered that in the interest of preserving the health of the consumers of such products it is necessary to incorporate some way to ensure that they are sanitized before reaching the consumer's hands.

Thus, it is then that an object of the invention that is proposed and that consists of a sanitizing device for vending machines to have appropriate means for the product to be sanitized inside the vending machine as well as means to specifically sanitize the product selected from the moment when it is selected to when it is received by the consumer.

### PRIOR ART

The inventor knows the germicidal properties of ultraviolet light which is used in bacterial control during the production, packaging and storage of food products. It is also used in hospitals, laboratories and places where it is required to minimize pollution and improve air quality. In the prior art, no realizations have been found that can be considered an obstacle to the present record.

### BRIEF DESCRIPTION OF THE INVENTION

As stated, the invention being described consists of a sanitizing device for vending machines that includes a microprocessor, interfaces, sensors, controls, UVC light emitters, controllers, sprayers and a display.

The invention may include some protective film to cover the transparent covers that are provided in some models of vending machines.

### FIGURES

It is accompanied by different schematic figures that allow a better understanding of the invention that is revealed here:

FIG. 1: schematizes a block diagram where the different components that make up the invention have been represented.

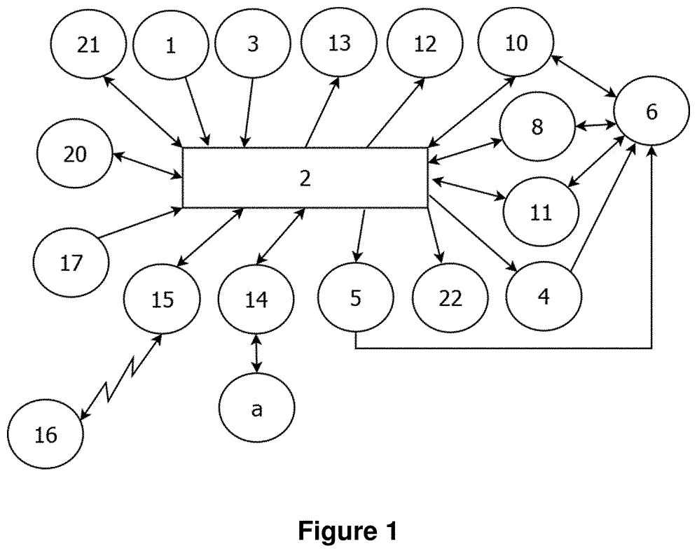

FIG. 2: schematizes a flow diagram where the different components of the invention are represented.

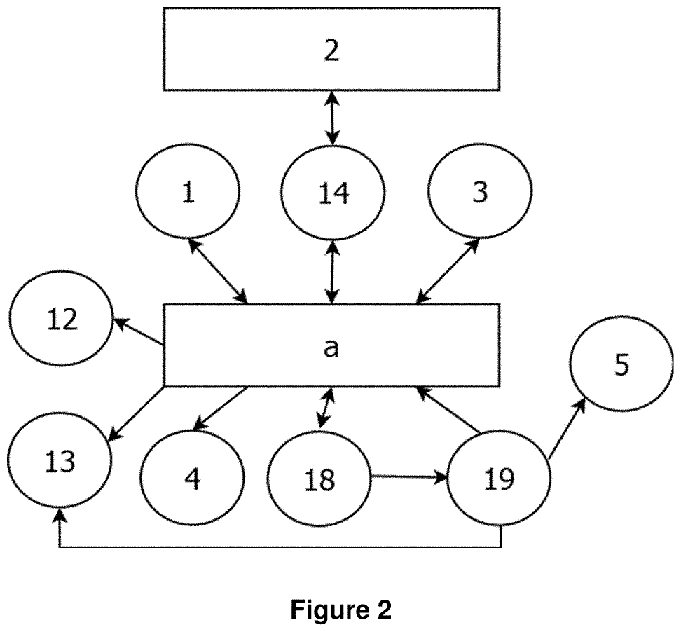

FIG. 3: schematizes a perspective view of a disinfection module. And finally,

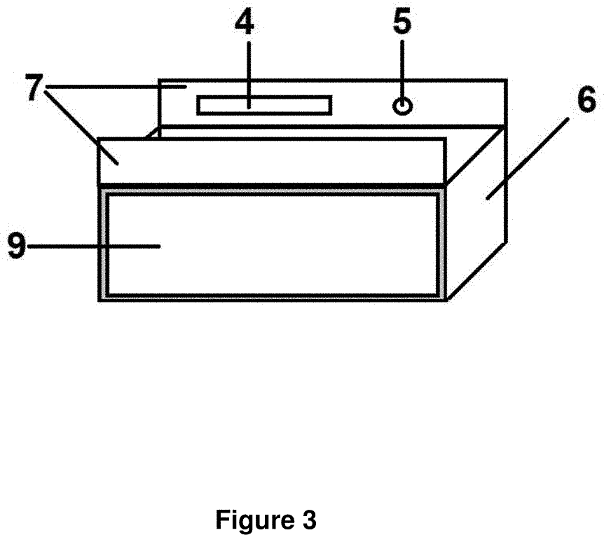

FIG. 4: schematizes a partial view of the vending machine keyboard, a UVC radiation emitter, an external radiation module and a pair of guides.

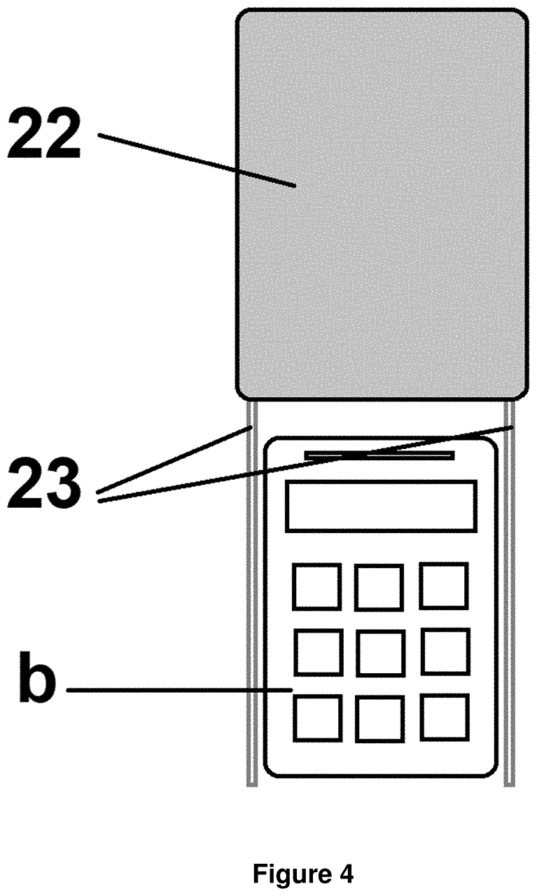

### REFERENCES

In the figures mentioned, the same characters represent equal or complementary parts.

It is clarified that the characters are put between periods the first time they are used and between parentheses in the subsequent opportunities. In this way, it is easier to follow the development of the writing since any reference between hyphens designates a part not previously described.

Thus, the reference 1 has been reserved to designate an opening sensor; reference 2 to designate a microcontroller; reference 3 to designate a proximity sensor; reference 4 to designate a radiation module; reference 5 to designate a sprayer; reference 6 to designate a disinfection compartment; reference 7 to designate an upper door; reference 8 to designate a control mechanism; reference 9 to designate a front door; the reference 10 to designate an opening and closing mechanism; reference 11 to designate a product sensor; reference 12 to designate a screen; the reference 13 to designate an external sprayer; reference 14 to designate a communication interface; reference 15 to designate a network communication interface; reference 16 to designate a remote location; reference 17 to designate a level sensor; reference 18 to designate a pump; reference 19 to designate a fluid reservoir; reference 20 to designate a pump controller; the reference 21 to designate a lighting controller of the radiation module; reference 22 to designate an external radiation module and reference 23 to indicate a pair of guides.

The letter -a- has been reserved to designate a vending machine or machine or vending machine and the letter -b- to designate a selection module.

### Functioning

Although there is a wide range of automatic vending machines, for the purposes of this description we can consider that we are faced with two large groups: the one that has part of the surface of the vending machine made of some transparent material, showing the products exhibited and the other group where the entire surface is opaque. Taking into account what is stated in the preceding paragraph, the present invention provides adaptable solutions for both groups.

The proposed invention has a disinfection means that is carried out by spraying a liquid sanitizing agent and a disinfection means based on ultraviolet radiation.

This disinfection ensures that the dispensed product reaches the consumer's hands free of viruses and bacteria and without altering the packaging or the products.

The present invention can be installed in existing vending machines or integrated into the manufacturing process in new vending machines.

The sanitizing device for vending machines that is disclosed allows disinfecting the products in two stages.

The first one is carried out by means of a UVC light radiation module or by means of the internal sprayers when access to the vending machine is closed once the products have been loaded or replaced.

The second stage is carried out during the time interval between the moment the product is required and the moment it is received by the consumer.

As will be understood, the solution is extremely efficient since the last disinfection is carried out in the instant prior to placing the product in contact with the consumer. Any contamination that could have affected the product during transport, during the resupply of the machine -a- or even after the replacement of products is finished, will be sanitized when the access to it is closed and will be reinforced in the moment before the delivery to the consumer.

To keep the vending machines stocked (a), the operator proceeds to open the supply door, closing it after restocking the merchandise.

Said supply door includes an opening sensor -1- that detects the event by sending a signal to a microcontroller 2 which awaits the closing signal of said supply door as well as the signal triggered by the proximity sensors 3.

This signal determines the presence, or not, of people in the area of the machine (a) sending said information, as already said, to the microcontroller 2 which, in the event of a signal indicating negative presence, the microcontroller 2 activates to a plurality of radiation modules 4 or to the sprayers 5 initiating a first disinfection cycle during a previously programmed period of time.

In a preferred implementation, the radiation modules (4) emit ultraviolet radiation in the 200 to 280 nm range, also known by its acronym UVC.

The microcontroller 2 will keep the radiation modules 4 and/or the sprays 5 on for the entire programmed time unless it receives a signal emitted by any of the proximity sensors 3 indicating the existence of living beings at radiation reach.

On the other hand, when a user selects a product among those offered by the vending machine (a), the mechanisms of the prior art machines follow a series of steps to deliver it to the final compartment.

In the case of the invention at hand, the product falls into a disinfection compartment -6- which is located before said final compartment or replaces it.

The disinfection compartment 6 has two upper doors 7 which are normally open to allow the product(s) selected by the consumer to pass through. Said upper doors 7 have a control mechanism 8 commanded by the microcontroller 2.

The disinfection compartment 6 also has a front door 9 normally closed and provided with a mechanism for opening and closing -10- also commanded by the microcontroller 2.

As already mentioned, the upper doors 7 are normally open to allow the entry of the product selected by the user into the disinfection compartment 6.

Once the product has entered into the disinfection compartment 6, a product sensor 11 detects it by sending a signal to the microcontroller 2 which activates the control mechanism 8 closing the upper doors 7 starting the second disinfection cycle during which, while the front door 9 is also closed, a totally isolated space is delimited to sanitize the selected product.

For this purpose, the microcontroller 2 activates, as appropriate, the radiation modules 4 or the sprayers 5, starting the second disinfection cycle.

In each of the six planes that make up the disinfection compartment 6 there is at least one radiation module 4 and at least one sprayer 5.

In the event that the radiation modules 4 are activated, which work with a wavelength of the order of 200 to 280 nm, they will expose the selected product to a higher dose of UVC radiation than that applied at the time of loading or reposition to which we already referred.

This higher radiation dose to the product is a consequence of, on the one hand, the greater power of the UVC emitters, on the other hand, as a consequence of the fact that they are closer to the product and finally that the radiation is applied from the six faces of the disinfection compartment 6.

Simultaneously, the microcontroller 2 emits an electronic indication to the processor of the vending machine (a) so that it reproduces on its display references to the sanitizing process that is being carried out in the disinfection compartment 6.

Considering that some vending machines (a) do not have a display, an alternative implementation has provided for the provision of a screen 12 to inform the consumer about the process.

After the programmed time to expose the product to disinfection, the microcontroller 2 turns off the disinfection medium and releases the front door 9 so that the consumer can remove the purchased product.

When the product is removed from the disinfection compartment 6 the product sensor 11 emits an empty compartment signal to the microcontroller 2 that acts on the opening and closing mechanism 10 closing the front door 9.

The microcontroller 2 also acts on the control mechanism 8, releasing the opening of the upper doors 8, thus leaving the disinfection compartment 6 ready to receive a new product when it is chosen.

As mentioned, during the disinfection cycle, the microcontroller 2 activates the nozzles 5 that spray disinfectant on the product entered into the disinfection compartment 6 or activate the radiation modules 4 that emit UVC radiation in a wavelength of between 200 and 280 nm.

The case may arise in which the vending machine (a) contains containers of different materials so the microcontroller 2, in accordance with the information received from the vending machine (a), will alternately activate the sprayers 5 or radiation modules 4 in order to use the most convenient method in accordance with the characteristics of the containers to be disinfected.

When referring to the characteristics of the containers to be disinfected, the material of the packaging or the product container is indicated.

Thus, if the product is packaged in cardboard or paper, it will be convenient to sanitize it using the radiation module 4, while if the container is made of plastic or metal material, the radiation module 4 or the sprayers 5.

After each operation carried out by a consumer, the microcontroller 2 activates the external sprayers 13 that spray disinfectant on the selection module -b- where the selection controls and the payment slots are included, and—if applicable—on the collection door.

The invention that is described includes a communication interface 14 that allows the link between the microcontroller 2 and the electronics of the vending machine (a) exchanging information about the product selection process, the synchronization to reproduce the audiovisual content in the display of the dispenser (a) or, if it is not available, on the incorporated screen 12.

The communication interface 14 also enables the vending machine (a) and the microcontroller 2 to exchange messages related to the control and maintenance thereof.

The invention also includes a network communication interface 15 that enables the microcontroller 2 to communicate with a remote location 16 to perform system configuration and update tasks.

A level sensor 17 detects the level of the disinfectant fluid contained in the respective tank generating, when it reaches a minimum, a signal that is sent to the microcontroller 2 which, through the communication interface 14 with the vending machine (a) and the network communication interface 15 sends a message requesting the replacement of the supply.

Considering that UVC radiation can be harmful to the user, it is advisable to place a film on the transparent areas of the vending machine (a) capable of blocking said radiation minimizing the risks.

The invention also includes a pump 18 for driving the sanitizing fluid from the fluid reservoir 19 to the sprayers 5; a pump control 20 and a radiation module lighting controller 21.

It has been stated that the microcontroller 2 activates external sprayers 13 which spray the disinfectant on the selection module (b) as well as the payment slots (slots). Furthermore, the machine (a) includes an external radiation module 22 provided with an insulating layer to protect the public from the radiation generated.

Said external radiation module 22 slides on guides 23 that allow it to move from a rest position to a working position from where to irradiate the selection buttons and the payment slots during a predetermined period.

Once this period has elapsed, the microcontroller 2 stops the emission of UVC radiation and emits a signal so that the radiation module moves along the guides 23, returning to the rest position after having left the selection module (b) free of contaminants, and suitable to be operated by a new user.

In this way, a preferred way of carrying out the invention has been described and illustrated, admitting also variants that are not limiting thereof.

Following is an attached statement establishing the novelty of the described invention; matter that is claimed as exclusive property.

1. A sanitizing device for vending machines, said machines having a supply door, a program, and a display, the sanitizing device comprising:
a microcontroller for receiving information from an opening sensor on a status of an access door to the interior of the vending machine, activating at least one radiation module or at least one sprayer for a first stage of sanitizing stored products;
wherein the microcontroller operates a control mechanism of an upper door and an opening and closing mechanism of a front door of a disinfection compartment closing both and starting a second stage of disinfection of the product selected by a consumer detected by a product sensor operating the opening of the front door at the end of the same, and wherein a signal of empty disinfection compartment triggers the opening and closing mechanism by closing the front door and the control mechanism by opening the upper door; wherein the presence detection sent by proximity sensors to the machine to the microcontroller, interrupts the cycle of ultraviolet radiation and whose microcontroller is linked to a communication interface and a network communication interface.

2. The device according to claim 1, wherein the disinfection compartment replaces the dispensing drawer of products and the collection door includes a container provided with two upper doors normally open and a front door normally closed; on each of the faces of said disinfection compartment there is at least one module of radiation, emitting ultraviolet radiation in a length between 200 and 280 nm and at least one sprayer of a sanitizing fluid.

3. The device in accordance with claim 1, wherein at the end of each operation, the microcontroller activates the external sprayers by spraying disinfectant fluid on the selection controls as well as on the payment slots and—if any—on the collection gate.

4. The device in accordance with claim 1, wherein at the end of each operation the microcontroller activates the external radiation module provided with an insulating layer and slidable on guides from a rest position to a work position, where it covers the selection module by radiating the selection buttons and the payment slots during a predetermined period of time after which the microcontroller cuts the UV radiation by retracting the external radiation module to its rest position.

5. The device in accordance with claim 1, wherein the measurement of the remaining volume of disinfectant fluid contained in the tank carried out by the level sensor is sent to the microcontroller whoso communication interface links it with the processor of the vending machine and, where appropriate, via the network communication interface with a remote location requesting the replacement of supplies and tasks of configuration and updating of the system.

6. The device in accordance with claim 1, wherein the communication interface links the microcontroller with the vending machine processor exchanging control and maintenance messages, product selection, synchronization of audiovisual content and reproduction in the display of the vending machine or on a screen.

7. The device in accordance with claim 1, wherein on the transparent areas of the vending a film capable of blocking UVC radiation is applied.

8. The device according to claim 1, wherein the radiation module is complemented by a lighting controller.

9. The device in accordance with claim 1, wherein a pump is provided to drive the sanitizing fluid from a tank to the sprayers.

10. The device in accordance with claim 4, wherein the pump includes a controller.
